(19) United States
(12) Patent Application Publication
(10) Pub. No.: US 2014/0363870 A1
(43) Pub. Date: Dec. 11, 2014

(54) PROCESS FOR OBTAINING COPPER NANOPARTICLES FROM RHODOTORULA MUCILAGINOSA AND USE OF RHODOTORULA MUCILAGINOSA IN BIOREMEDIATION OF WASTEWATER AND PRODUCTION OF COPPER NANOPARTICLES

(71) Applicants: VALE S.A., Rio de Janeiro (BR); Universidade de São Paulo - USP, Sao Paulo (BR)

(72) Inventors: Benedito CORRÈA, Sao Paulo (BR); Cláudio Augusto Oller NASCIMENTO, Sao Paulo (BR); Márcia Regina SALVADORI, Salto (BR)

(21) Appl. No.: 14/297,379

(22) Filed: Jun. 5, 2014

Related U.S. Application Data
(60) Provisional application No. 61/831,357, filed on Jun. 5, 2013.

(51) Int. Cl. C12P 3/00; C22C 9/00; C02F 1/28

(52) U.S. Cl. CPC C12P 3/00; C02F 1/286; C22C 9/00; C02F 2101/20
USPC 435/168; 210/660; 420/469

(57) ABSTRACT

The present invention refers to a process for obtaining copper nanoparticles from Rhodotorula mucilaginosa.

The present invention refers to the use of dead biomass of Rhodotorula mucilaginosa to perform bioremediation of wastewater and for industrial scale production of copper nanoparticles.

In the present invention, it is developed a synthetic strategy for the biosynthesis and removal of copper nanoparticles which is fast, low cost, environment friendly and easily scalable, using as a reduction agent the yeast Rhodotorula mucilaginosa.

### FIELD OF THE INVENTION

The present invention refers to a process for obtaining copper nanoparticles from Rhodotorula mucilaginosa.

The present invention refers to the use of dead biomass of Rhodotorula mucilaginosa, to perform bioremediation of copper-containing wastewater, in order to produce copper nanoparticles. The invention allows producing copper nanoparticles in industrial scale.

### BACKGROUND OF THE INVENTION

Heavy metals are the major contaminants in rivers and industrial effluents. To be very reactive and bioaccumulative element in living organisms, heavy metals have received special attention, since some are extremely toxic even in very low amounts, for instance chromium, cadmium and mercury. The use of fungi and yeasts in the removal or reduction of these pollutants is an environmentally suitable alternative, since the environmental impact caused by these types of remediation is small.

Recently, synthesis of inorganic nanoparticles has been demonstrated by many physical and chemical means. But the importance of biological synthesis is being emphasized globally at present because chemical methods are capital intensive toxic, non-ecofriendly and have low productive [Singh A V, Patil R, Anand A, Milani P, Gade W N (2010) Biological synthesis of copper oxide nanopaticles using Escherichia coli. CurrNanosci 6: 365-369]. Copper nanoparticles, due to their unique physical and chemical properties and the low cost of preparation, have been of great interest recently. Furthermore, copper nanoparticles have potential industrial use such as gas sensors, catalytic processes, high temperature superconductors, solar cells and so on [Li Y, Liang J, Tao Z, Chen J (2007) CuO particles and plates: Synthesis and gas-sensor application. Mater Res Bull 43: 2380-2385; Guo Z, Liang X, Pereira T, Scaffaro R, Hahn H T (2007) CuO nanoparticle filled vinyl-ester resin nanocomposites: Fabrication, characterization and property analysis. Compos Sci Tech 67: 2036-2044].

New alternatives for the synthesis of metallic nanoparticles are currently being explored through bacteria, fungi, yeast and plants [Bharde A A, Parikh R Y, Baidakova M, Jouen S, Hannoyer B, Enoki T, et al. (2008) Bacteria-mediated precursor-dependent biosynthesis of super paramagnetic iron oxide and iron sulfide nanoparticles. Langmuir 24: 5787-5794; Lang C, Schüler D, Faivre D (2007) Synthesis of magnetite nanoparticles for bio-and nanotechnology: genetic engineering and biomimetics of bacterial magnetosomes. MacromolBiosci 7: 144-151]. Wastewater from copper mining often contain a high concentration of this toxic metal generated during the extraction, beneficiation, and processing of metal. In recent years, the bioremediation, through of the biosorption of toxic metals as copper has received a great deal of attention not only as a scientific novelty, but also because of its potential industrial applications.

This novel approach is competitive, effective, and cheap [Volesky B (2001) Detoxification of metal bearing effluents: biosorption for the next century. Hydrometallurgy 59: 203-216]. In this respect, fungi have been used in bioremediation processes since they are a versatile group that can adapt to and grow under various extreme conditions of pH, temperature and nutrient availability, as well as at high concentrations of metals [Anand P, Isar J, Saran S, Saxena R K (2006) Bioaccumulation of copper by Trichoderma viride. Bioresource Technol 97: 1018-1025]. Consequently, there has been considerable interest in developing biosynthesis methods for the preparation of copper nanoparticles as an alternative to physical and chemical methods.

Literature review of previous studies revealed that few articles were published on biosynthesis of copper nanoparticles [Varshney R, Bhadauria S, Gaur M S (2012) A review: Biological synthesis of silver and copper nanoparticles. Nano Biomed Eng 4: 99-106] and none of the studies used the yeast Rhodotorula mucilaginosa (R. mucilaginosa). Also, most of the biosynthesis studies on copper nanoparticles focused on bioreduction phase only and ignored the important biosorption phase of the process.

Studying towards the goal to enlarge the scope of biological systems for the biosynthesis of metallic nanomaterials and bioremediation of wastewater, it is explored for the first time the use of the yeast R. mucilaginosa, to the uptake and reduction of copper ions to copper nanoparticles. Thus, the bioremediation and green synthesis of copper nanoparticles, has been achieved in the present study using dead biomass of R. mucilaginosa.

### BRIEF DESCRIPTION OF THE FIGURES

FIG. 1 shows Batch biosorption studies. Influence of the physico-chemical factors on the live and dead biomass of R. mucilaginosa. (A) Effect of the amount of biosorbent. (B) Effect of pH. (C) Effect of temperature. (D) Effect of contact time. (E) Effect of agitation rate. (F) Effect of initial copper concentration.

FIG. 2 shows Biosorption isotherm models and biosorption kinetics of R. mucilaginosa. Langmuir plots for live (A) and dead (B) biomass. Pseudo second-order models for live (C) and dead biomass (D).

FIG. 3 shows TEM micrographs of R. mucilaginosa sections. (A) before contact with the metal ion showing the cell wall, cytoplasmic membrane and cytoplasm with no metal , and (B) after contact with the metal ion copper showing the nanoparticles (darkest arrow) accumulated intracellularly and cell wall (arrow clearer).

FIG. 4 shows Dead biomass of R. mucilaginosa analyzed by SEM-EDS. (A) Control (without copper) and (B) biomass exposed to copper.

FIG. 5 shows EDS spectra recorded of dead biomass of R. mucilaginosa. (A) before exposure to copper solution and (B) after exposure to copper

FIG. 6 shows FTIR spectra of dead biomass of R. mucilaginosa. (A) before and (B) after to saturation with copper ions.

### SUMMARY OF THE INVENTION

The present invention refers to a process for obtaining copper nanoparticles from Rhodotorula mucilaginosa.

The present invention refers to the use of dead biomass of Rhodotorula mucilaginosa to perform bioremediation of wastewater and for industrial scale production of copper nanoparticles.

### DETAILED DESCRIPTION OF THE INVENTION

A biological system for the biosynthesis of nanoparticles and uptake of copper from wastewater using dead biomass of R. mucilaginosa was analyzed and described for the first time.

In the present invention, it is explored for the first time the intracellularly biosynthesis and uptake of copper nanoparticles from wastewater utilizing the dead biomass of the yeast R. mucilaginosa.

In the present invention, it is developed a synthetic strategy for the biosynthesis and removal of copper nanoparticles which is fast, low cost, environment friendly and easily scalable, using as a reduction agent the yeast R. mucilaginosa.

The present invention refers to a process for obtaining copper nanoparticles from R. mucilaginosa comprising the following steps:
a. Isolation of the fungus R. mucilaginosa;
b. Determination of copper tolerance of the isolated fungus of step a;
c. Preparation of a copper stock solution;
d. Addition of said isolated fungus in the medium culture YEPD broth resulting in a live biomass;
e. Subjecting the live biomass to autoclave resulting in a dead biomass; and
f. Determination of copper nanoparticles retention in the live and dead biomass.

The determination of copper retention by biosorption of the isolated fungus is performed by addition for each one of the biomasses (live and dead) in a copper solution item [0020] step c;

The biosorption of copper onto dead and live biomass of fungus was performed in function of the: initial metal concentrations (25-600 mg L⁻¹), pH (2-6), temperature (20-60° C.), agitation (50-250 rpm), inoculum volume (0.05-0.75 g) and contact time (5-360 min).

The development of the invention will be illustrated by the following no-exhaustive examples.

### BRIEF SUMMARY OF THE TESTS AND RESULTS

The equilibrium and kinetics investigation of the biosorption of copper onto dead and live biomass of yeast was performed in function of the initial metal concentration, pH, temperature, agitation and inoculum volume.

The range of biosorption capacity of cooper was observed for dead biomass, completed within 60 min of contact, at pH 5.0, temperature of 30° C., at agitation speed of 150 rpm with a maximum biosorption of copper of 20-35 mg g⁻¹.

The equilibrium data were better described using the Langmuir isotherm and Kinetic analysis indicated the pseudo-second-order model. The average size, morphology and location of nanoparticles biosynthesized by the yeast were determined by scanning electron microscopy (SEM), energy dispersive X-ray spectroscopy (EDS) and transmission electron microscopy (TEM).

The shape of nanoparticles was found to be mainly spherical with an average size of 5-25 nm and synthesized intracellularly. Fourier transform infrared spectroscopy (FTIR) with Attenuated total reflectance (ATR) study disclosed revealed that the observed differences in the spectra of dead biomass after contact with the copper are very subtle, since almost all the copper nanoparticles were internalized and few of the nanoparticles bound extracellularly, probably through carboxyl groups, whose vibrational frequency showed a slight variation.

These studies demonstrate that dead biomass of R. mucilaginosa offers an economical and technically feasible option for bioremediation of wastewater and for industrial scale production of copper nanoparticles.

### 1. Growth and Maintenance of the Organism

R. mucilaginosa was isolated from the water collected from a pond of copper waste from Sossego mine, located in Canã dos Carajás, Pará, Brazilian Amazonia region (06° 26′ S latitude and 50° 4′ W longitude). R. mucilaginosa was maintained and activated in YEPD agar medium (10 g yeast extract L⁻¹, 20 g peptone L⁻¹, 20 g glucose L⁻¹ and 20 g agar L⁻¹) media compounds were obtained from Oxoid (England) [Machado M D, Soares E V, Soares H M V M (2010) Removal of heavy metals using a brewer's yeast strain of Saccharomyces cerevisiae: Chemical Speciation as a tool in the prediction and improving of treatment efficiency of real electroplating effluents. J Hazard Mater 180: 347-353].

### 2. Minimum Inhibitory Concentration in Agar Medium

Copper tolerance of the isolated yeast was determined as the minimum inhibitory concentration (MIC) by the spot plate method. YEPD agar medium plates containing different concentrations of copper (50 to 3000 mg L⁻¹) were prepared and inocula of the tested yeast were spotted onto the metal and control plates (plate without metal) [Ahmad I, Ansari M I, Aqil F (2006) Biosorption of Ni, Cr and Cd by metal tolerante Aspergillus niger and Penicillium sp using single and multi-metal solution. Indian J Exp Biol 44: 73-76]. The plates were incubated at 25° C. for at least 5 days. The MIC is defined as the lowest concentration of metal that inhibits visible growth of the isolate.

### 3. Determination of Copper Nanoparticles Retention by the Biosorbent

### 3.1. Preparation of the Adsorbate Solutions

All chemicals used in the present study were of analytical grade and were used without further purification. All dilutions were prepared in double-deionized water (Milli-Q Millipore 18.2 Ωcm⁻¹ conductivity). The copper stock solution was prepared by dissolving CuCl₂.2H₂O (Carlo Erba, Italy) in double-deionized water. The working solutions were prepared by diluting this stock solution.

### 3.2. Biomass Preparation

The fungal biomass was prepared in the YEPD broth (10 g yeast extract L⁻¹, 20 g peptone L⁻¹, 20 g glucose L⁻¹), and incubated at 25° C. for 5 days, at 150 rpm. After incubation, the pellets were harvested and washed with of double-deionized water this was referred to as live biomass. For the preparation of dead biomass, an appropriate amount of live biomass was autoclaved [Salvadori M R, Ando R A, do Nascimento C A O, Corrêa B (2014) Intracellular biosynthesis and removal of copper nanoparticles by dead biomass of yeast isolated from the wastewater of a mine in the Brazilian Amazonia. Plos One 9: 1-9].

### 3.3. Studies of the Effects of Physico-Chemical Factors on the Efficiency of Adsorption of Copper Nanoparticles by the Biosorbent

The pH (2-6), temperature (20-60° C.), contact time (5-360 min), initial copper concentration (25-600 mg L⁻¹), and agitation rate (50-250 rpm) on the removal of copper was analysed. Such experiments were optimized at the desired pH, temperature, metal concentrations, contact time, agitation rate and biosorbent dose (0.05-0.75 g) using 45 mL of 100 mg L⁻¹ of Cu (II) test solution in plastic flask.

Several concentrations (25-600 mg g⁻¹) of copper (II) were prepared by appropriate dilution of the copper (II) stock solution. The pH was adjusted with HCl or NaOH. The desired biomass dose was then added and the content of the flask was shaken for the desired contact time in an electrically thermostatic reciprocating shaker at the required agitation rate. After shaking, the Cu (II) solution was separated from the biomass by vacuum filtration through a Millipore membrane. The metal concentration in the filtrate was determined by flame atomic absorption spectrophotometer (AAS). The efficiency (R) of metal removal was calculated using following equation:

R=(Cᵢ−Cₑ)/Cᵢ·100

where Cᵢ and Cₑ are initial and equilibrium metal concentrations, respectively. The metal uptake capacity, qₑ, was calculated using the following equation:

qₑ=V(Cᵢ−Cₑ)/M

where qₑ(mg g⁻¹) is the biosorption capacity of the biosorbent at any time, M (g) is the biomass dose, and V (L) is the volume of the solution.

### 3.4. Biosorption Isotherm Models

Biosorption was analyzed by the batch equilibrium technique using the following sorbent concentrations of 25-600 mg L⁻¹. The equilibrium data were fit using Freundlich and Langmuir isotherm models [Volesky B (2003) Biosorption process simulation tools. Hydrometallurgy 71: 179-190]. The linearized Langmuir isotherm model is:

Cₑ/qₑ=1/(qₘ·b)+Cₑ/qₘ

where qₘ is the monolayer sorption capacity of the sorbent (mg g⁻¹), and b is the Langmuir sorption constant (L mg⁻¹). The linearized Freundlich isotherm model is:

lnqₑ=lnK_{F}+1/n·lnCₑ

where K_{F} is a constant relating the biosorption capacity and 1/n is related to the adsorption intensity of adsorbent.

### 3.5. Biosorption Kinetics

The results of rate kinetics of Cu (II) biosorption were analyzed using pseudo-first-order, and pseudo-second-order models. The linear pseudo-first-order model can be represented by the following equation [Lagergren S (1898) About the theory of so called adsorption of soluble substances. Kung Sven Veten Hand 24: 1-39]:

log(qₑ−qₜ)=logqₑ−K₁/2.303·t

where, qₑ (mg g⁻¹) and qₜ(mg g⁻¹) are the amounts of adsorbed metal on the sorbent at the equilibrium time and at any time t, respectively, and K₁ (min⁻¹) is the rate constant of the pseudo-first-order adsorption process. The linear pseudo-second-order model can be represented by the following equation [Ho Y S, Mckay G (1999) Pseudo-second-order model for sorption process. Process Biochem 34: 451-465]:

t/qₜ=1/K₂·qₑ²+t/qₑ

where K₂ (g mg⁻¹ min⁻¹) is the equilibrium rate constant of pseudo-second-order.

### 4. Biosynthesis of Metallic Copper Nanoparticles by R. mucilaginosa

In this study was used only the dead biomass of R. mucilaginosa that showed a high adsorption capacity of copper metal ion compared to live biomass. Biosynthesis of copper nanoparticles by dead biomass of R. mucilaginosa was investigated using the data of the equilibrium model at a concentration of 100 mg L⁻¹ of copper (II) solution.

### 4.1. TEM Observation

Analysis by Transmission electron microscopy (TEM) was used for determining the size, shape and location of copper nanoparticles on biosorbent, where cut ultra-thin of the specimens, were observed in a transmission electron microscope (JEOL-1010).

### 4.2. SEM-EDS Analysis

Analysis of small fragments of the biological material before and after the formation of copper nanoparticles, was performed on pin stubs and then coated with gold under vacuum and were examined by SEM on a JEOL 6460 LV equipped with an energy dispersive spectrometer (EDS).

### 4.3. FTIR-ATR Analysis

Infrared vibrational spectroscopy (FTIR) was used to identify the functional groups present in the biomass and to evaluate the spectral variations caused by the presence of copper nanoparticles. The infrared absorption spectra were obtained on Bruker model ALPHA interferometric spectrometer. The samples were placed directly into the sample compartment using an attenuated total reflectance accessory of single reflection (ATR with Platinum-crystal diamond). Eighty spectra were accumulated for each sample, using spectral resolution of 4 cm⁻¹.

R. mucilaginosa, isolated from copper mine, was subjected to minimum inhibitory concentration (MIC) at different copper concentrations (50-3000 mg L⁻¹) and the results indicated that R. mucilaginosa exhibited high tolerance to copper (2000 mg L⁻¹).

### 4.4. Influence of the Physico-Chemical Factors on Biosorption

The present investigation showed that copper removal by R. mucilaginosa biomass was influenced by physico-chemical factors such as biomass dosage, pH, temperature, contact time, rate of agitation and metal ion concentration. The biosorbent dose is an important parameter since it determines the capacity of a biosorbent for a given initial concentration of the metals.

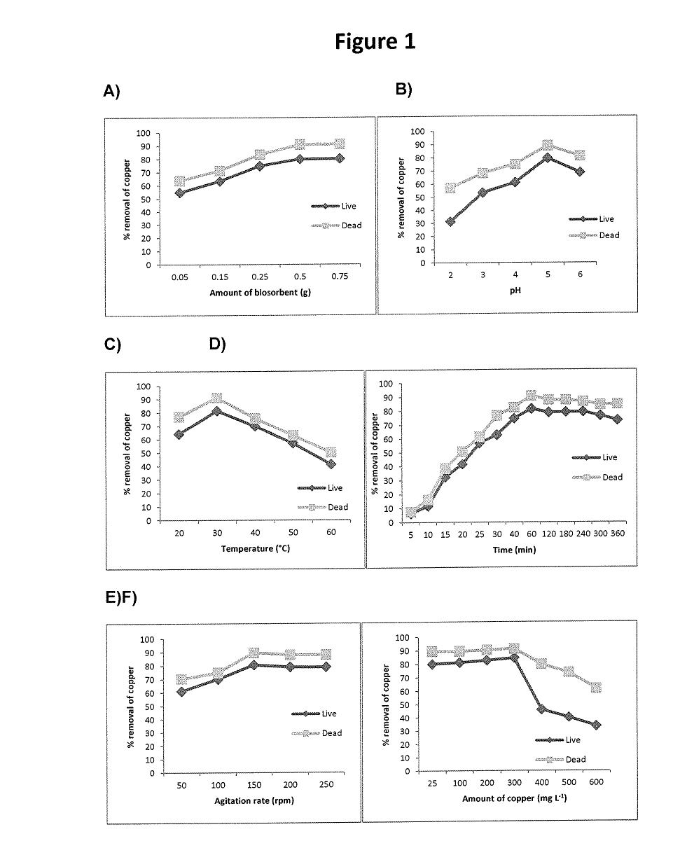

As shown in FIG. 1(A) the removal of copper by dead and live biomass by R. mucilaginosa recorded an increase with increase in the concentration of biomass and reached saturation at 0.75 g L⁻¹. The percent removal of copper by dead biomass was greater than live biomass FIG. 1(A). The dead biomass for Cu (II) removal offers advantages: the metal removal system is not subjected to toxicity and does not require growth media or nutrients. Maximum removal of copper was observed at pH 5.0 for the two types of biomass as shown in FIG. 1B. At lower pH value, the cell wall of R. mucilaginosa becomes positively charged and it is responsible for reduction in biosorption capacity. In contrast, at higher pH (pH 5), the cell wall surface becomes more negatively charged and therefore the biosorption of Cu (II) onto R. mucilaginosa is high due to attraction between the biomass and the positively charged metal ion.

The maximum removal of copper was observed at 30° C. for the two types of biomass (FIG. 1C). The effect of the temperature on biosorption of the metal suggested an interaction between the metal and the ligands on the cell wall. It is observed that the graph (FIG. 1D) follows the sigmoid kinetics which is characteristic of enzyme catalysis reaction for both types of biomass. The kinetics of copper nanoparticles formation to dead biomass showed that more than 90% of the particles were formed within the 60 min of the reaction, which suggests that the formation of copper nanoparticles is exponential. The optimum copper removal was observed at an agitation speed of 150 rpm for both types of biomass (FIG. 1E). At high agitation speeds, vortex phenomena occur and the suspension is no longer homogenous, a fact impairing metal removal [Liu Y G, Fan T, Zeng G M, Li X, Tong Q, et al. (2006) Removal of cadmium and zinc ions from aqueous solution by living Aspergillus niger. Trans Nonferrous Met Soc China 16: 681-686].

The percentage of copper adsorption decreased with increasing metal concentration (25-600 mg L⁻¹) at the two types of biomass as shown in FIG. 1F.

### 4.5. Sorption Isotherm and Kinetics Models

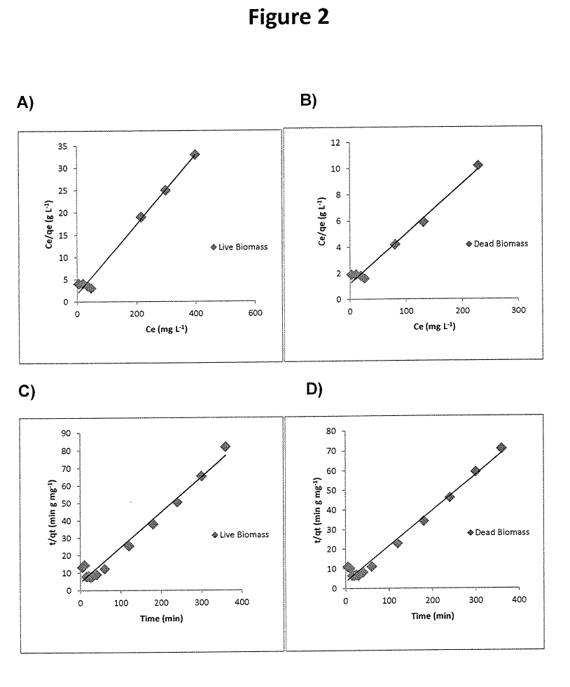

The Langmuir and Freundlich isotherm models were used to fit the biosorption data and to determine biosorption capacity. The Langmuir isotherm for Cu (II) biosorption obtained of the two types of R. mucilaginosa biomass is shown in FIG. 2A and FIG. 2B. The isotherm constants, maximum loading capacity estimated by the Langmuir and Freundlich models, and regression coefficients are shown in Table 1. The Langmuir model better described the Cu (II) biosorption isotherms than the Freundlich model. The maximum adsorption rate of Cu (II) by R. mucilaginosa (26.2 mg g⁻¹) observed in this study was similar or higher than the adsorption rates reported for other known biosorbents, such as Pleurotus pulmonaris, Schizophyllum commune, Penicillium spp, Rhizopus arrhizus, Trichoderma viride, Pichia stipitis, Pycnoporussanguineus with adsorption rates of 6.2, 1.52, 15.08, 19.0, 19.6, 15.85 and 2.76 mg g⁻¹ respectively [Veit M T, Tavares C R G, Gomes-da-Costa S M, Guedes T A (2005) Adsorption isotherms of copper (II) for two species of dead fungi biomasses. Process Biochem 40: 3303-3308; Du A, Cao L, Zhang R, Pan R (2009) Effects of a copper-resistant fungus on copper adsorption and chemical forms in soils. Water Air Soil Poll 201: 99-107; Rome L, Gadd D M (1987) Copper adsorption by Rhizopus arrhizus, Cladosporium resinae and Penicillium italicum. Appl Microbiol Biotechnol 26: 84-90; Kumar B N, Seshadri N, Ramana D K V, Seshaiah K, Reddy A V R (2011) Equilibrium, Thermodynamic and Kinetic studies on Trichoderma viride biomass as biosorbent for the removal of Cu (II) from water. Separ Sci Technol 46: 997-1004 Yilmazer P, Saracoglu N (2009) Bioaccumulation and biosorption of copper (II) and chromium (III) from aqueous solutions by Pichia stiptis yeast. J Chem Technol Biot 84: 604-610; Yahaya Y A, Matdom M, Bhatia S (2008) Biosorption of copper (II) onto immobilized cells of Pycnoporus sanguineus from aqueous solution: Equilibrium and Kinetic studies. J Hazard Mater 161: 189-195].

Comparison with biosorbents of bacterial origin showed that the Cu (II) adsorption rate of R. mucilaginosa is comparable to that of Bacillus subtilis IAM 1026 (20.8 mg g⁻¹) [Nakajima A, Yasuda M, Yokoyama H, Ohya-Nishiguchi H, Kamada H (2001) Copper sorption by chemically treated Micrococcus luteus cells. World J Microb Biot 17: 343-347], and compared with the algae the yeast R. mucilaginous also showed a high rate of adsorption of metal ion higher algae Cladophora spp and Fucusvesiculosus (14.28 and 23.4 mg g⁻¹) [Elmacy A, Yonar T, Özengin N (2007) Biosorption characteristics of copper (II), chromium (III), nickel (II) and lead (II) from aqueous solutions by Chara sp and Cladophora sp. Water Environ Res 79: 1000-1005; Grimm A, Zanzi R, Björnbom E, Cukierman A L (2008) Comparison of different types of biomasses of copper biosorption. Bioresource Technol 99: 2559-2565]. The kinetics of Cu (II) biosorption onto both types of biomass of R. mucilaginosa were analysed using pseudo-first-order and pseudo-second-order models. All the constants and regression coefficients are shown in Table 2. In the present study, biosorption by R. mucilaginosa was best described using a pseudo-second-order kinetic model as shown in FIG. 2C and FIG. 2D. This adsorption kinetics is typical for the adsorption of divalent metals onto biosorbents [Reddad Z, Gerent C, Andres Y, LeCloirec P (2002) Adsorption of several metal ions onto a low-cost biosorbents: kinetic and equilibrium studies. Environ Sci Technol 36: 2067-2073].

### 4.6. Biosynthesis of Copper Nanoparticles

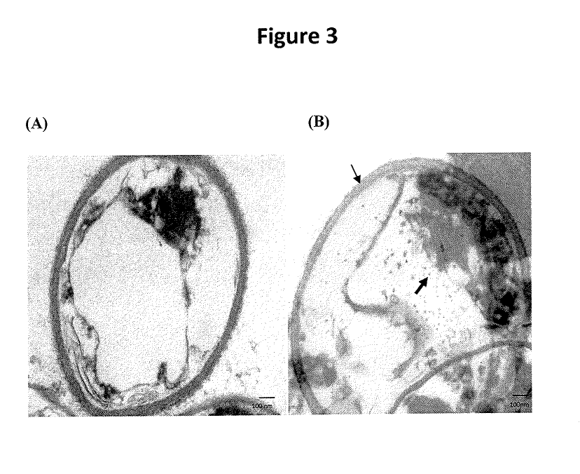

The studying of the involved mechanisms of the nanoparticles formation by biological systems is important in order to determine even more reliable and reproducible methods for its biosynthesis. To understanding the formation of nanoparticles in fungal biomass, was examined by TEM a fraction of the dead biomass. The location of the nanoparticles in R. mucilaginosa was investigated and the electron micrograph revealed that mostly of the nanoparticles were found intracellularly, and was absent in control, the ultrastructural change such as shrinking of cytoplasmatic material was observed in control and biomass impregnated with copper due to autoclaving process (FIG. 3A and FIG. 3B). The shape and size of nanoparticles are two of the most important features controlling the physical, chemical, optical and electronic properties of the nanoscopic materials [Alivisatos A P (1996) Perspectives on the physical chemistry of semiconductor nanocrystals. J Phys Chem 100: 13226-13239; Aizpurua J, Hanarp P, Sutherland D S, Käll M, Bryant G W, et al. (2003) Optical properties of gold nanorings. Phys Rev Lett 90: 57401-57404].

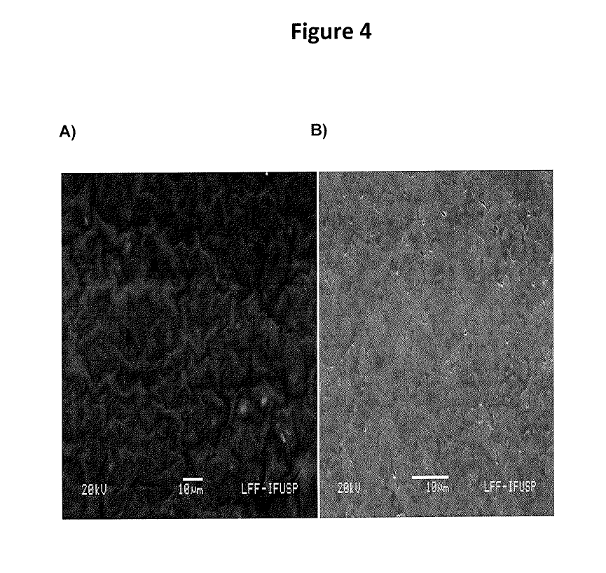

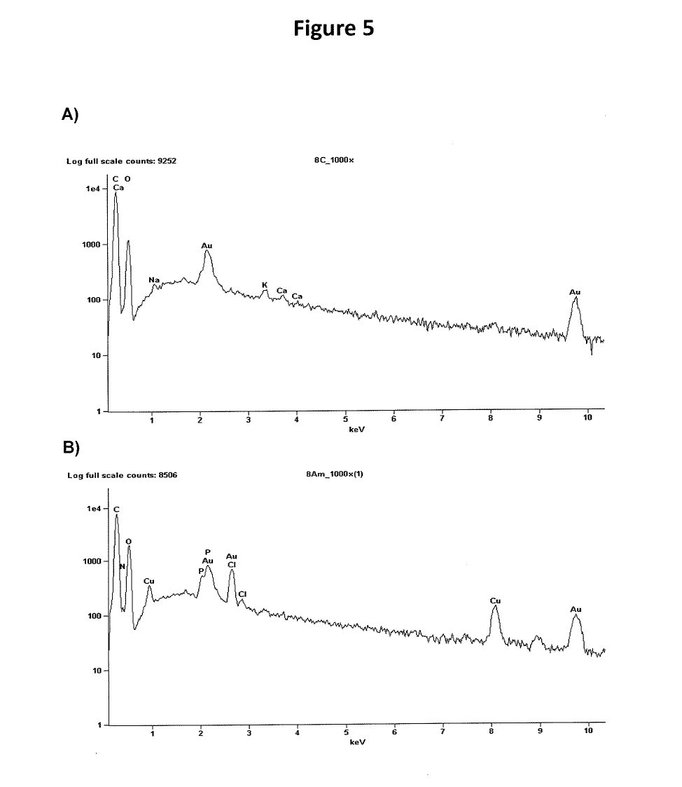

In this study copper nanoparticles showed an average diameter of 10.5 nm (FIG. 3B). The presence of copper nanoparticles was confirmed by spot profile SEM-EDS measurement. SEM micrographs recorded before and after biosorption of Cu (II) by fungal biomass was presented in FIG. 4A and FIG. 4B respectively. We observed that a surface modification occurred by increasing the irregularity, after binding of copper nanoparticles onto the surface of the fungus biomass. EDS spectra recorded in the examined region of the yeast, show signals from copper (FIG. 5A and FIG. 5B) for the yeast.

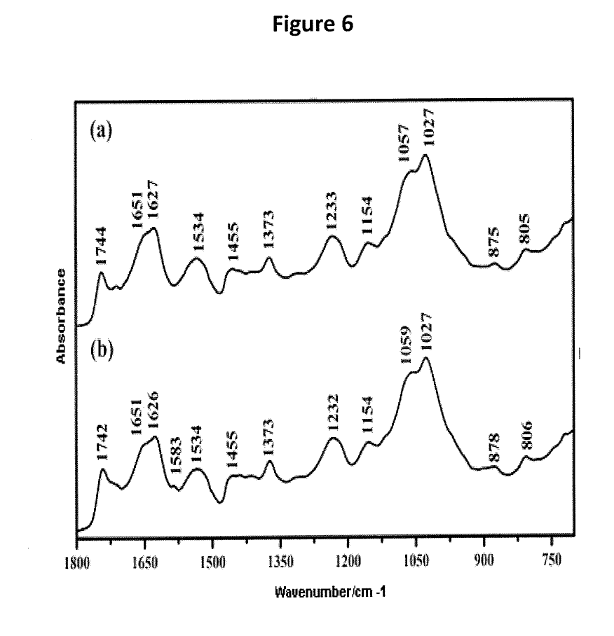

In this study, FT-IR revealed that the observed differences in the spectra of dead biomass after contact with the copper are very subtle, since almost all the copper nanoparticles were internalized and few of the nanoparticles bound extracellularly, probably through carboxyl groups, whose vibrational frequency showed a slight variation. The bands at 1744 and 1057 cm⁻¹ were shifted to 1742 and 1059 cm⁻¹, respectively (FIG. 6). As previously mentioned, in R. mucilaginosa copper nanoparticles were found accumulated within the cell yeast, probably the reduction process inside the cell was carried out by protein and enzymes present in the cytoplasm [Sanghi R, Verma P (2009) Biomimetic synthesis and characterization of protein capped silver nanoparticles. Bioresource Technol 100: 501-504]. However, the type of protein involved in interactions with nanoparticles of copper which was studied remains to be determined. Such understanding may lead to a more efficient green process for the production of copper nanoparticles.

TABLE 1
Adsorption constants from simulations with
Langmuir and Freundlich models.

| Type of | Langmuir model | | | Freundlich model | | |
|---|---|---|---|---|---|---|
| biomass | qₘ(mg g⁻¹) | b (L mg⁻¹) | R² | K_{F} (mg g⁻¹) | 1/n | R² |
| Live | 12.7 | 0.046 | 0.988 | 0.59 | 0.44 | 0.641 |
| Dead | 26.3 | 0.031 | 0.984 | 0.74 | 0.61 | 0.850 |

TABLE 2
Kinetic parameters for adsorption of copper.

| | | |
|---|---|---|
| Type of | Pseudo-first-order | Pseudo-second-order |

| biomass | K₁ (min⁻¹) | R² | K₂ (g mg⁻¹ min⁻¹) | R² |
|---|---|---|---|---|
| Live | 7.36 × 10⁻³ | 0.474 | 9.45 × 10⁻³ | 0.972 |
| Dead | 6.90 × 10⁻³ | 0.502 | 9.69 × 10⁻³ | 0.981 |

1. PROCESS FOR OBTAINING COPPER NANOPARTICLES from Rhodotorula mucilaginosa comprising the following steps:
a. Isolation of the yeast Rhodotorula mucilaginosa;
b. Determination of copper tolerance of the isolated fungus of step a;
c. Preparation of a copper stock solution;
d. Addition of said isolated fungus in the medium culture YEPD broth resulting in a live biomass;
e. Subjecting the live biomass to autoclave resulting in a dead biomass; and
f. Determination of copper nanoparticles retention in the live and dead biomass.

2. USE OF A YEAST EXTRACT, selected from Rhodotorula mucilaginosa extract to perform bioremediation of wastewater.

3. THE USE, according to claim 2, wherein Rhodotorula mucilaginosa extract is dead mass of Rhodotorula mucilaginosa.

4. THE USE, according to one of the claims 1 to 3, wherein it is for the production of copper nanoparticles.

5. COPPER NANOPARTICLE, produced from a yeast selected Rhodotorula mucilaginosa during a bioremediation of wastewater.
